(19) United States
(12) Patent Application Publication
(10) Pub. No.: US 2024/0225973 A1
(43) Pub. Date: Jul. 11, 2024

(54) COSMETIC COMPOSITION FOR IMPROVING SENSITIVE SKIN

(71) Applicant: LG HOUSEHOLD & HEALTH CARE LTD., Seoul (KR)

(72) Inventors: Yumi SHIM, Seoul (KR); Sangkyung KIM, Seoul (KR); Jinyong LEE, Seoul (KR); Ohsun KWON, Seoul (KR)

(73) Assignee: LG HOUSEHOLD & HEALTH CARE LTD., Seoul (KR)

(21) Appl. No.: 18/410,924

(22) Filed: Jan. 11, 2024

(30) Foreign Application Priority Data
Jan. 11, 2023 (KR) 10-2023-0004062

(51) Int. Cl. A61K 8/35; A61K 8/46; A61K 8/64; A61Q 19/00

(52) U.S. Cl. CPC A61K 8/35; A61K 8/466; A61K 8/64; A61Q 19/005; A61K 2800/74

(57) ABSTRACT

The present disclosure relates to a cosmetic composition and a quasi-drug composition, each for protecting or improving sensitive skin, soothing the skin, or strengthening the skin barrier, the cosmetic composition and quasi-drug composition comprising curcumin in combination with taurine and/or acetyl hexapeptide-8 as active ingredients.

### TECHNICAL FIELD

The present disclosure relates to a composition for protecting or improving sensitive skin, soothing the skin, or strengthening the skin barrier, comprising curcumin in combination with taurine and/or acetyl hexapeptide-8 as active ingredients.

### BACKGROUND ART

As the number of consumers who feel that their skin is sensitive is increasing after the coronavirus, the demand for hypoallergenic products is growing. However, simple products for sensitive skin with previous moisturizing and soothing effects are being released without any understanding of sensitive skin, and thus there is no difference from previous products. Therefore, it is necessary to find ingredients capable of reducing sensitivity, based on an accurate understanding of sensitive skin.

Sensitive skin has the feature of feeling tingling, burning, and stinging sensations more easily than normal skin. There are many different factors that cause this sensation, such as chemicals, mental stress, hormones, temperature changes, wind, etc. The skin has systems to respond to these irritants, and one of the systems that respond first is defensin, which is a defense protein on the skin surface, and this protein can be reduced by stress factors.

Modern people are continuously exposed to external stimulating factors, but they are also continuously exposed to mentally stressful situations. Further, research has shown that when cortisol secretion increases due to mental stress, defensin decreases, which is a defense protein on the skin surface. Defensin is a defense peptide that responds to bacterial or viral invasion. In addition to its defensive role, it is also involved in skin regeneration and involved in repairing damaged barriers.

There are several types of defensins, which are broadly divided into alpha and beta. In the skin, beta-defensin 1 to 3 are best known. Each peptide plays a slightly different role, and defensin 1 and 3 play a role in helping to synthesize proteins that close the gaps in the skin barrier. Defensin 2 acts as a danger-detecting signal to recruit various other cells, and participates in cell migration and proliferation to help the wound area to quickly regenerate, which can help restoration of damaged barriers. Among them, human beta-defensin 2 (hBD-2) is an antimicrobial peptide which is produced by epithelial cells in response to microorganisms or inflammation, and hBD-2 enhances cell-mediated immune responses, and also has the function of promoting wound healing by influencing proliferation and differentiation processes of epidermal cells and fibroblasts (Park Young-do, 2007.12., Increased expression of Human β-defensin-2 by TNF-α in HaCaT keratinocytes).

Under this background, in the present disclosure, effective substances enhancing beta-defensin 2, which is reduced by a stress hormone cortisol, was found through evaluations. Among these substances, a combination of curcumin and taurine and a combination of curcumin and acetyl hexapeptide-8 (AHP-8) were found to have synergistic effects. Accordingly, it was confirmed that combinations of the above substances may be used as a material for protecting or improving sensitive skin, for soothing the skin, and for improving the barrier.

### DISCLOSURE

### Technical Problem

The technical problem of the present application is to provide a composition for protecting or improving sensitive skin, soothing the skin, or strengthening the skin barrier, comprising curcumin in combination with taurine and/or acetyl hexapeptide-8 as active ingredients.

### Technical Solution

An object of the present disclosure is to provide a composition for protecting or improving sensitive skin, soothing the skin, or strengthening the skin barrier, comprising curcumin in combination with taurine and/or acetyl hexapeptide-8 as active ingredients.

Another object of the present disclosure is to provide a method of protecting, improving, or treating sensitive skin, soothing the skin, or strengthening the skin barrier, comprising applying to the skin of a subject in need thereof, the composition of the present disclosure.

Another object of the present disclosure is to provide a method of protecting, improving, or treating sensitive skin, soothing the skin, or strengthening the skin barrier, comprising administering to a subject in need thereof, the composition of the present disclosure.

### Advantageous Effects

In the present disclosure, synergistic effects on enhancing the synthesis of beta-defensin 2 were observed in combinations of curcumin; and taurine and/or acetyl hexapeptide-8, and the combinations may be used as a cosmetic composition or quasi-drug composition, which is excellent in protecting or improving sensitive skin, soothing the skin, and strengthening the skin barrier.

### BRIEF DESCRIPTION OF THE DRAWING

FIG. 1 shows effects of enhancing beta-defensin 2 synthesis by intracellular treatment with curcumin, taurine, or acetyl hexapeptide-8 alone, which are expressed by regarding a value of a cortisol treatment group as 100.

FIG. 2 shows effects of enhancing beta-defensin 2 synthesis by intracellular cotreatment with curcumin and taurine, which are expressed by regarding a value of a cortisol treatment group as 100.

FIG. 3 shows effects of enhancing beta-defensin 2 synthesis by intracellular cotreatment with curcumin and acetyl hexapeptide-8, which are expressed by regarding a value of a cortisol treatment group as 100.

FIG. 4 shows effects of enhancing beta-defensin 2 synthesis by intracellular cotreatment with curcumin, taurine, and acetyl hexapeptide-8, which are expressed by regarding a value of a cortisol treatment group as 100.

FIG. 5 shows results of comparison of the amount of beta-defensin 2 synthesis in the human skin between a control group and cotreatment groups of curcumin; and taurine and/or acetyl hexapeptide-8.

FIG. 6 shows results of comparison of the erythema index after barrier damage in the human skin between a control group and cotreatment groups of curcumin and taurine.

FIG. 7 shows results of comparison of the erythema index after barrier damage in the human skin between a control group and cotreatment groups of curcumin and acetyl hexapeptide-8.

FIG. 8 shows results of comparison of the erythema index after barrier damage in the human skin between a control group and cotreatment groups of curcumin, taurine and acetyl hexapeptide-8.

### DETAILED DESCRIPTION OF PREFERRED EMBODIMENTS

The present disclosure will be described in detail as follows. Meanwhile, each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description described below.

Further, those skilled in the art will recognize, or be able to ascertain using no more than routine experimentation, many equivalents to the specific embodiments of the disclosure described herein. Further, these equivalents should be interpreted to fall within the present disclosure.

An aspect of the present disclosure provides a composition for protecting or improving sensitive skin, soothing the skin, or strengthening the skin barrier, comprising curcumin in combination with taurine and/or acetyl hexapeptide-8 as active ingredients.

Specifically, in the present disclosure, the composition may include curcumin and taurine as active ingredients, may include curcumin and acetyl hexapeptide as active ingredients, or may include curcumin, taurine, and acetyl hexapeptide as active ingredients.

As used herein, the “curcumin” refers to a type of polyphenol mainly contained in Indian turmeric, and its chemical formula is C₂₁H₂₀O₆. The curcumin is known to have anti-tumor, antioxidant, anti-amyloid and anti-inflammatory effects, but is not limited thereto.

As used herein, the “taurine”, which is an organic substance having a structure of 2-aminoethanesulfonic acid, mainly refers to a sulfur-containing amine present in the cells and tissues of mammals including humans, but is not limited thereto.

As used herein, the “acetyl hexapeptide-8” refers to a peptide fragment of SNAP-25 which is a substrate for botulinum toxin which is one of the toxins that paralyze muscles. It is known to regulate the secretion of acetylcholine by competitively acting with SNAP-25 protein, but is not limited thereto.

As used herein, the “sensitive skin” refers to skin that reacts more sensitive to environmental changes, such as external irritants or allergic substances, as compared to normal skin, and is prone to irritant reactions or dermatitis. There are very diverse and complex causes of sensitive skin. Sensitive skin may occur innately due to genetic factors, or may occur by external factors such as harmful substances, stress, climate, seasonal changes, and work environment. The main causes of sensitive skin include excessive neurological responses, increased immune responses, damage to the skin barrier, etc. Most cases are observed due to damage to the skin barrier, but are not limited thereto. In addition, sensitive skin is accompanied by symptoms such as erythema, dry skin, stinging, itching, burning and inflammation, etc., but is not limited thereto.

A representative example of sensitive skin is atopic skin, and research has revealed that defensin is reduced in atopic skin compared to normal skin. The defensin is known to have an antimicrobial effect of removing external harmful substances and to be also involved in the regeneration of skin barrier damage. In particular, the defensin is known to play a role in maintaining homeostasis of the skin barrier by promoting the synthesis of proteins which constitute tight junctions in keratinocytes, such as claudin and occludin (Herman, A. & Herman, A. P., 2019, Antimicrobial peptides activity in the skin, Ski. Res. Technol. 25, 111-117; Hai Le Thanh Nguyen, 2020, Role of Antimicrobial peptides in skin barrier repair in individuals with atopic dermatitis, Int. J. Mol. Sci. 21(20), 7607).

As used herein, “protecting sensitive skin” means all actions by which occurrence of sensitive skin is retarded or symptoms such as erythema, skin dryness, stinging, itching, burning and inflammation due to the occurrence of sensitive skin are retarded by the administration of the composition. The protecting sensitive skin may be achieved by strengthening the skin barrier by enhancing the synthesis of beta-defensin 2.

As used herein, “improving sensitive skin” means all actions by which occurrence of sensitive skin is reduced, or the degree of symptoms such as erythema, skin dryness, stinging, itching, burning and inflammation due to the occurrence of sensitive skin is reduced, or the damage skin is restored. The improving sensitive skin may be achieved by strengthening the skin barrier or by soothing the skin by enhancing the synthesis of beta-defensin 2.

As used herein, “soothing the skin” means alleviating and calming erythema or irritated skin areas, etc., and may be a concept including relieving skin irritation. Examples may include reducing moisture loss and/or redness of the skin, but are not particularly limited thereto.

As used herein, “skin barrier” refers to the outermost stratum corneum of the skin, which plays a role in maintaining skin moisture and protecting the skin from external stimuli. As used herein, “strengthening the skin barrier” includes all actions that strengthen the skin barrier, or repair or regenerate a damaged barrier, which may be attributed to enhancement of the synthesis of defensin 2, which functions in the skin barrier, but is limited thereto. In addition, by strengthening the skin barrier, a barrier layer may be served against the outside, such as protecting moisture loss from the epidermal stratum corneum to the outside and controlling the entry and exit of substances from the outside, but is not limited thereto.

The composition may promote the synthesis of beta-defensin 2 (β-defensin 2). As used herein, “defensin” refers to a peptide that consists of amino acids containing cysteine secreted as a defense mechanism of host cells, when infected with a pathogen. It is known that defensin not only plays a defensive role but also participates in skin regeneration to be involved in repairing damaged barriers, but is not limited thereto. Defensin is widely found in vertebrates, invertebrates, plants, and fungi, and there are several types of defensins, which are largely divided into alpha-defensins and beta-defensins, but are not limited thereto. Among them, beta-defensin 2 (human β-defensin 2, hBD-2) refers to an antimicrobial peptide which is produced by epithelial cells in response to microorganisms or inflammation, and it is known that hBD-2 mainly enhances cell-mediated immune responses, and has the function of promoting wound healing by influencing proliferation and differentiation processes of epidermal cells and fibroblasts, but is not limited thereto.

In the present disclosure, the composition may specifically comprise curcumin in combination with taurine or acetyl hexapeptide-8 at a weight ratio of 1:1000 to 100:1, preferably, at a weight ratio of 1:100 to 10:1, but is not limited thereto.

Further, the composition may specifically comprise curcumin, taurine, and acetyl hexapeptide-8 at a weight ratio of 1 to 1000:1 to 1000:1 to 1000, preferably, at a weight ratio of 1 to 500:1 to 500:1 to 500, and more preferably, at a weight ratio of 1 to 100:1 to 100:1 to 100, but is not limited thereto.

Further, the composition may comprise curcumin, taurine, and acetyl hexapeptide-8 each being in an amount of 0.00001% by weight to 10% by weight, preferably, in an amount of 0.001% by weight to 10% by weight, respectively, based on the total weight, but is not limited thereto.

Specifically, in the present disclosure, the composition may be a cosmetic composition or a quasi-drug composition.

As used herein, the “cosmetic composition” is basically applied to the skin, and thus it may be prepared into any formulation commonly prepared with reference to cosmetic compositions in the art. For example, the cosmetic composition may be prepared into a formulation selected from the group consisting of a solution, an external ointment, a cream, a foam, a nourishing lotion, a softening lotion, a pack, an emulsion, a makeup base, a foundation, an essence, a soap, a liquid cleanser, a bath salt, a sunscreen, a sun oil, a suspension, a gel, a lotion, a powder, a surfactant-containing cleanser, a patch, and a spray, but is not limited thereto.

In addition to curcumin, taurine, and acetyl hexapeptide-8 of the present disclosure, the cosmetic composition may further include common auxiliaries and carriers, such as antioxidants, stabilizers, solubilizers, vitamins, pigments, fragrances, etc., which are commonly used in cosmetic compositions. For example, the cosmetic composition may further include auxiliary ingredients such as glycerin, butylene glycol, polyoxyethylene hydrogenated castor oil, tocopheryl acetate, citric acid, panthenol, squalane, sodium citrate, allantoin, etc.

The cosmetic composition may be formulated into a softening lotion, an astringent lotion, a nourishing lotion, a nourishing cream, a massage cream, an essence, a pack, a skin adhesive patch, a skin adhesive gel, a powder, an ointment, a paste, a gel, a suspension, an emulsion, a spray, a cosmetic liquid, or a capsule, but is not particularly limited thereto.

The cosmetic composition of the present disclosure may further include one or more cosmetically acceptable carriers which are blended in general skin cosmetics, and common ingredients may be appropriately blended with, for example, oil, water, surfactants, moisturizers, alcohol, thickeners, chelating agents, pigments, preservatives, fragrances, etc., but is not limited thereto. The cosmetologically acceptable carriers included in the cosmetic composition of the present disclosure vary depending on the formulation.

When the formulation of the presented disclosure is an ointment, a paste, a cream, or a gel, animal oil, vegetable oil, wax, paraffin, starch, tracant, cellulose derivatives, polyethylene glycol, silicone, bentonite, silica, talc, zinc oxide, or a mixture thereof may be used as the carrier ingredient.

When the formulation of the present disclosure is a powder or a spray, lactose, talc, silica, aluminum hydroxide, calcium silicate, polyamide powder, or a mixture thereof may be used as the carrier ingredient, and in particular, when it is a spray, propellants such as chlorofluorohydrocarbons, propane/butane, or dimethyl ether may be further included.

When the formulation of the present disclosure is a solution or an emulsion, a solvent, a solubilizing agent, or an emulsifying agent, for example, water, ethanol, isopropanol, ethyl carbonate, ethyl acetate, benzyl alcohol, benzyl benzoate, propylene glycol, or 1,3-butyl glycol oil may be used as the carrier ingredient. Particularly, cottonseed oil, peanut oil, corn germ oil, olive oil, castor oil and sesame oil, glycerol aliphatic esters, polyethylene glycol, or sorbitan fatty acid esters may be used.

When the formulation of the present disclosure is a suspension, a liquid diluent such as water, ethanol, or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar or tracant, etc. may be used as the carrier ingredient.

Meanwhile, when the formulation of the present disclosure is a capsule, it may be formulated in the form of an alginate capsule, an agar capsule, a gelatin capsule, a wax-like capsule, or a double capsule, but is not particularly limited thereto.

As used herein, the term “quasi-drug” refers to articles which have a milder action than a drug among articles used for the purpose of diagnosing, treating, ameliorating, alleviating, treating, or protecting a human or animal disease, and for example, according to the Pharmaceutical Affairs Act, the quasi-drug is an article excluding articles which are used for pharmaceutical purposes, and includes products used for the treatment or prevention of human/animal diseases, products that have little or no direct effect on the human body. In the present disclosure, the quasi-drug composition may have the use in protecting or improving sensitive skin, soothing the skin, or strengthening the skin barrier.

The “quasi-drug composition” may further include pharmaceutically acceptable carriers, excipients, or diluents as needed, in addition to the above ingredients. The pharmaceutically acceptable carriers, excipients, or diluents are not limited as long as they do not impair the effect of the present disclosure, and may include, for example, fillers, extenders, binders, wetting agents, disintegrants, surfactants, lubricants, sweeteners, fragrances, preservatives, etc. Specifically, the quasi-drug may include skin preparations for external use, and personal hygiene products. More specifically, the quasi-drug may be a disinfectant cleanser, a shower foam, a mouthwash, a wet tissue, a detergent soap, a hand wash, or an ointment, but is not limited thereto.

When the composition according to the present disclosure is used as a quasi-drug additive, the composition may be added as it is or may be used with other quasi-drugs or quasi-drug ingredients, and may be appropriately used according to common methods. The mixing amount of the active ingredients may be appropriately determined depending on the purpose of use.

Still another aspect of the present disclosure provides a method of protecting, improving, or treating sensitive skin, soothing the skin, or strengthening the skin barrier, comprising applying to the skin of a subject in need thereof, the composition comprising curcumin in combination with taurine and/or acetyl hexapeptide-8 as active ingredients.

Still another aspect of the present disclosure provides a method of protecting, improving, or treating sensitive skin, soothing the skin, or strengthening the skin barrier, comprising administering to a subject in need thereof, the composition comprising curcumin in combination with taurine and/or acetyl hexapeptide-8 as active ingredients.

Still another aspect of the present disclosure provides use of a composition comprising curcumin in combination with taurine and/or acetyl hexapeptide-8 as active ingredients in protecting or improving sensitive skin, soothing the skin, or strengthening the skin barrier.

### MODE FOR CARRYING OUT THE INVENTION

Hereinafter, the present disclosure will be described in more detail with reference to exemplary embodiments. However, these exemplary embodiments are only for illustrating the present disclosure, and the scope of the present disclosure is not intended to be limited by these exemplary embodiments.

### Example 1. Cell Culture and Sample Treatment Method

First, HaCaT, which is a human epidermal cell of the outermost layer among skin cells, was cultured using Dulbecco's Modified Eagle's Media (DMEM; Gibco, USA) mixed with 10% fetal bovine serum (FBS; Gibco, USA) and 1% antibiotic antimycotic (Gibco, USA) in a 24-well plate under conditions of 37° C. and 5% CO₂.

After cells reached 80% or more confluence, they were treated with 5 mM of a stress hormone cortisol (hydrocortisone) (Sigma Aldrich, USA) to reduce the synthesis of beta-defensin 2 (human β-defensin 2) and cultured for 24 hours. At this time, it was confirmed that beta-defensin 2 was significantly reduced by cortisol in skin cells.

After 24 hours, the cells were treated with curcumin, taurine, and acetyl hexapeptide-8 at experimental concentrations, and cultured for 24 hours. At this time, curcumin, taurine, and acetyl hexapeptide-8 were treated alone, and the concentration of curcumin was fixed at 0.1 ppm and 1 ppm, and then taurine and acetyl hexapeptide-8 were treated in combination of concentrations, and cultured for 24 hours.

### Example 2. Quantification Method

24 hours after treatment with each sample, ELISA was performed to quantify the amount of beta-defensin 2 (human β-defensin 2, hBD-2) protein synthesis. ELISA was performed using the HUMAN BETA DEFENSIN 2 ELISA kit (ALPHA DIAGNOSTIC, USA) and using the cell culture medium according to the kit's manual, and the final protein amount was calculated by measuring absorbance at 400 nm.

The amount of beta-defensin 2 in the untreated group was regarded as 100%, and the amount of beta-defensin 2 after sample treatment was quantified. At this time, when the amount thereof increased compared to the decrease due to cortisol, it was interpreted that the synthesis of beta-defensin 2 was increased by the sample.

In addition, the synergistic effect between respective active substances that enhance beta-defensin 2 which had been reduced by cortisol was predicted through the Colby formula, and when the actual measured value was greater than the predicted value, it was determined that there was the synergistic effect.

### Example 3. Examination of Effect of Enhancing Beta-Defensin 2 Synthesis by Curcumin, Taurine, and Acetyl Hexapeptide-8 (In Vitro Experiment)

### Example 3-1. Results of Single Treatment with Curcumin, Taurine, or Acetyl Hexapeptide-8

To confirm the synergistic effect of each sample, 0.1 ppm to 1 ppm of curcumin, 0.01 ppm to 10 ppm of taurine, and 0.01 ppm to 10 ppm of acetyl hexapeptide-8 (AHP-8) were treated alone, and the efficacy was examined.

TABLE 1

| | | | Production amount of |
|---|---|---|---|
| | Applied concentration | | beta-defensin 2 (%) |
| Cortisol (control) | 5 | μM | 100.00 |
| Curcumin | 0.01 | ppm | 96.65 |
| | 0.1 | ppm | 97.92 |
| | 1 | ppm | 106.82 |
| AHP-8 | 0.01 | ppm | 98.45 |
| | 0.1 | ppm | 101.37 |
| | 1 | ppm | 98.75 |
| | 5 | ppm | 110.12 |
| | 10 | ppm | 92.66 |
| Taurine | 0.01 | ppm | 100.60 |
| | 0.1 | ppm | 99.90 |
| | 1 | ppm | 117.60 |
| | 5 | ppm | 87.61 |
| | 10 | ppm | 83.06 |

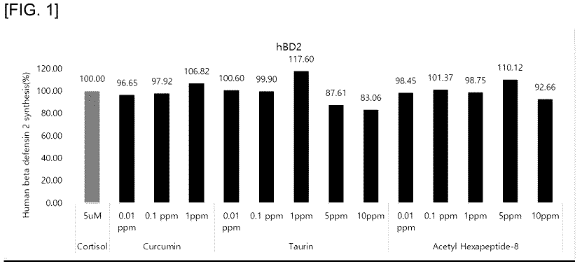

As a result, as shown in Table 1 and FIG. 1, the enhancement efficacy of curcumin and taurine was confirmed at 1 ppm, and the enhancement efficacy of acetyl hexapeptide was confirmed at 0.1 ppm and 5 ppm.

### Example 3-2. Results of Cotreatment with Curcumin and Taurine

When the concentration of curcumin was fixed at 0.1 ppm and 1 ppm and taurine was cotreated at 0.1 ppm to 10 ppm, respectively, the efficacy of enhancing beta-defensin 2 synthesis was examined. At this time, the enhancement efficacy was predicted through the Colby formula using the results of the evaluation of single treatment in Example 3-1. The Colby formula used to calculate the predicted value is as follows.

$$Colby formula ( E ) = X + Y - ( X * Y / 100 )$$

TABLE 2

| | | Production amount of |
|---|---|---|
| | Applied concentration | beta-defensin 2 (%) |
| Curcumin 0.1 ppm | Taurine 0.1 ppm | 148.74 |
| | Taurine 1 ppm | 156.46 |
| | Taurine 10 ppm | 508.43 |
| Curcumin 1 ppm | Taurine 0.1 ppm | 129.08 |
| | Taurine 1 ppm | 119.21 |
| | Taurine 10 ppm | 118.04 |

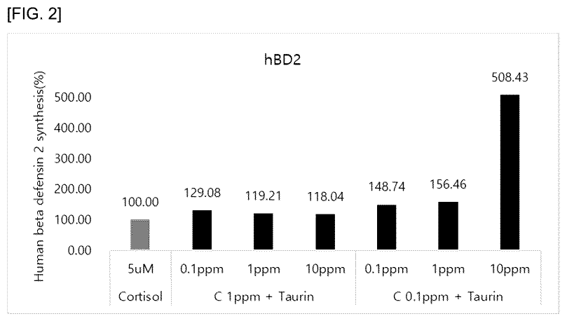

As a result, as shown in Table 2 and FIG. 2, it was confirmed that all the actual efficacy was measured to be higher than the predicted value according to the Colby formula. Therefore, it was found that there was a synergistic effect on enhancing beta-defensin 2 when curcumin and taurine were treated in combination below.

### Example 3-3. Results of Cotreatment with Curcumin and Acetyl Hexapeptide-8

When the concentration of curcumin was fixed at 0.1 ppm and 1 ppm and acetyl hexapeptide-8 (AHP-8) was cotreated at 0.1 ppm to 10 ppm, respectively, the efficacy of enhancing beta-defensin 2 synthesis was examined. At this time, the enhancement efficacy was predicted through the Colby formula using the results of the evaluation of single treatment in Example 3-1. The Colby formula used to calculate the predicted value is as follows.

$$Colby formula ( E ) = X + Y - ( X * Y / 100 )$$

TABLE 3

| | | Production amount of |
|---|---|---|
| | Applied concentration | beta-defensin 2 (%) |
| Curcumin 0.1 ppm | AHP-8 0.1 ppm | 101.72 |
| | AHP-8 1 ppm | 109.32 |
| | AHP-8 10 ppm | 105.69 |
| Curcumin 1 ppm | AHP-8 0.1 ppm | 101.99 |
| | AHP-8 1 ppm | 115.56 |
| | AHP-8 10 ppm | 155.77 |

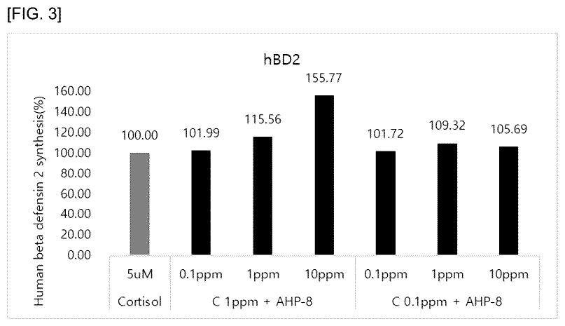

As a result, as shown in Table 3 and FIG. 3, it was confirmed that all the actual efficacy was measured to be higher than the predicted value according to the Colby formula. Therefore, it was found that there was a synergistic effect on enhancing beta-defensin 2 when curcumin and acetyl hexapeptide-8 were used in combination.

### Example 3-4. Results of Cotreatment with Three of Curcumin, Taurine, and Acetyl Hexapeptide-8

The efficacy of enhancing beta-defensin 2 synthesis was examined, when three of curcumin, taurine, and acetyl hexapeptide-8 (AHP-8) were cotreated. At this time, the enhancement efficacy was predicted through the Colby formula using the results of the evaluation of single treatment in Example 3-1. The Colby formula used to calculate the predicted value is as follows.

$$Colby formula E = X + Y + Z - \{ ( XY + XZ + YZ ) / 100 ) \} + ( XYZ / 10000 )$$

TABLE 4

| | Production amount of |
|---|---|
| Sample | beta-defensin 2 (%) |
| Cortisol (5 μM) | 100 |
| Curcumin (1 ppm) | 106.82 |
| Taurine (1 ppm) | 117.6 |
| AHP-8 (1 ppm) | 98.75 |
| Curcumin (1 ppm) + Taurine (1 ppm) + | 125 |
| AHP-8 (1 ppm) | |

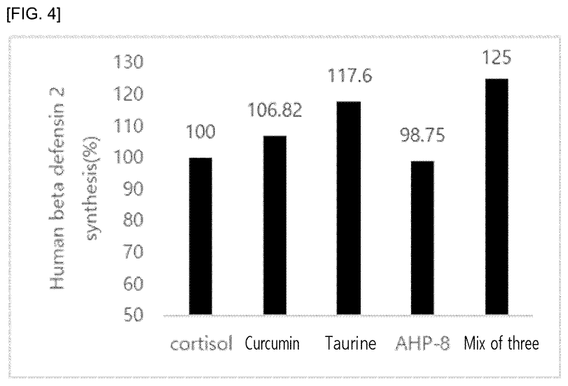

As a result, as shown in Table 4 and FIG. 4, it was confirmed that the value measured as the actual efficacy was higher than the predicted value according to the Colby formula. Therefore, it was found that the combination of curcumin, taurine, and acetyl hexapeptide-8 has a synergistic effect on enhancing beta-defensin 2.

### Example 4. Examination of Effect of Enhancing Beta-Defensin 2 Synthesis and Effect of Reducing Erythema by Curcumin, Taurine, and Acetyl Hexapeptide-8 (In Vivo Experiment)

### Example 4-1. In Vivo Experimental Method

Through a test on humans, it was examined whether beta-defensin 2 was enhanced in actual skin. First, the area to be evaluated was determined on the forearm of 10 subjects, and then 1% SLS was used to damage the barrier. A formulation containing a mixture of curcumin and taurine at 1:100 was applied for 4 weeks, and dead skin cells were collected from the damaged area using a patch. In the case of curcumin and acetyl hexapeptide-8, a 1:10 mixed formulation was equally applied, and dead skin cells were collected from the damaged area using a patch.

After separating the proteins from the collected dead skin cells, defensin 2 ELISA was performed in the same manner as the in vitro method to compare the difference in beta-defensin 2 production depending on whether or not the formulation was applied.

Further, erythema of the test area was measured using a mexameter (Mexameter® MX 18 (Courage Khazaka, Germany)).

### Example 4-2. Results of In Vivo Experiment

TABLE 5

| | Production amount of |
|---|---|
| Sample | beta-defensin 2 (%) |
| Control | 100 |
| Curcumin + Taurine | 131.2958 |
| Curcumin + AHP-8 | 141.8478 |
| Curcumin + Taurine + AHP-8 | 158.0838 |

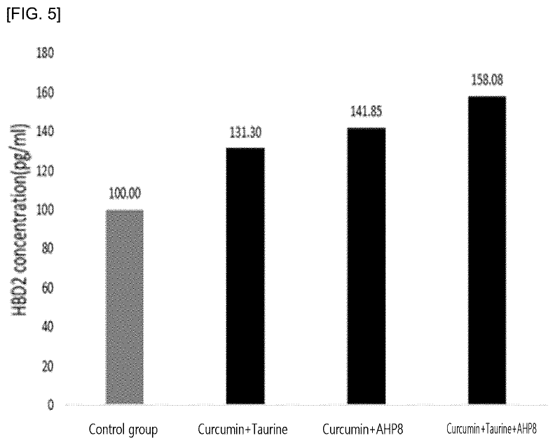

As a result of the experiment, as shown in Table 5 and FIG. 5, it was observed that the amount of beta-defensin 2 more increased in the areas where products of each combination were used, as compared to the control group. Accordingly, it was confirmed that the effect of each combination of curcumin; and taurine and/or acetyl hexapeptide-8 on enhancing beta-defensin 2 synthesis, shown in vitro, was also observed in the test on humans.

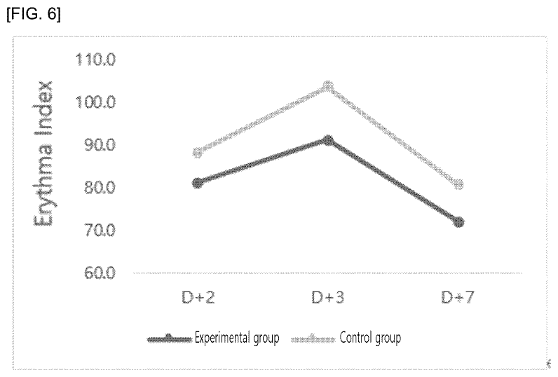

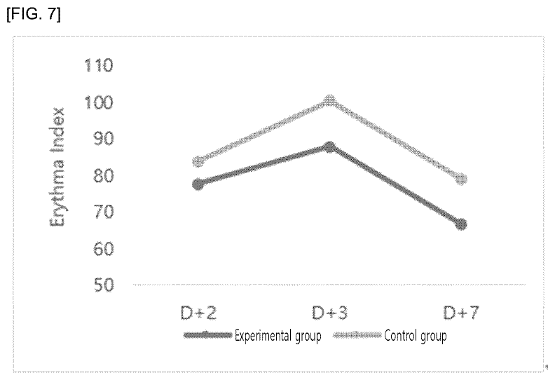

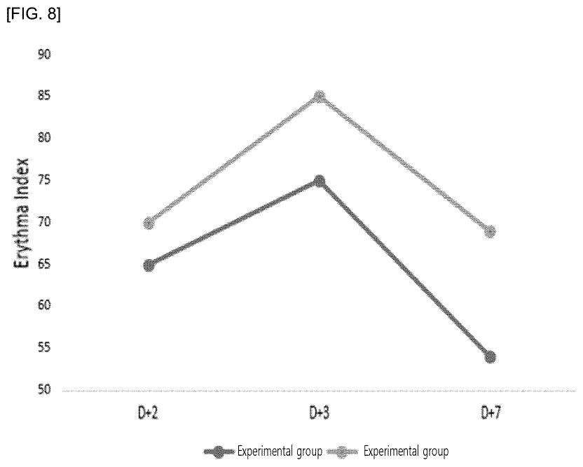

Further, graphs of FIGS. 6 to 8 sequentially show the results of measuring erythema in the same areas after the mixed products of curcumin and taurine; curcumin and acetyl hexapeptide-8; curcumin, taurine, and acetyl hexapeptide-8 were applied, and it was confirmed that all three combinations showed less erythema in the areas where the products were applied, as compared to the control group.

Accordingly, it was found that the combination products of curcumin; and taurine and/or acetyl hexapeptide-8 may enhance the synthesis of beta-defensin 2, may help improvement of barrier damage, and may be particularly effective in improving erythema.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds are therefore intended to be embraced by the claims.

1. A composition for protecting or improving sensitive skin, soothing the skin, or strengthening the skin barrier, comprising curcumin in combination with taurine and/or acetyl hexapeptide-8 as active ingredients.

2. The composition of claim 1, wherein the composition promotes beta-defensin 2 (β-defensin 2) synthesis.

3. The composition of claim 1, wherein the composition comprises curcumin in combination with taurine or acetyl hexapeptide-8 at a weight ratio of 1:1000 to 100:1.

4. The composition of claim 1, wherein the composition comprises curcumin in combination with taurine and acetyl hexapeptide-8 each being in an amount of 0.00001% by weight to 10% by weight based on the total weight of the composition.

5. The composition of claim 1, wherein the composition is a cosmetic composition comprising a cosmetically acceptable carrier, or a quasi-drug composition comprising a pharmaceutically acceptable carrier.

6. A method of protecting, improving, or treating sensitive skin, soothing the skin, or strengthening the skin barrier, comprising applying to the skin of a subject in need thereof, the composition of claim 1.

7. A method of protecting, improving, or treating sensitive skin, soothing the skin, or strengthening the skin barrier, comprising administering to a subject in need thereof, the composition of claim 1.
